SEQUENCE LISTING (4 sequences)

SEQ ID NO: 1 - protein, 6 aa, synthetic construct
NPNNXN

SEQ ID NO: 2 - protein, 25 aa, synthetic construct
XXXXXXXXXXXXXNPNNXNXXXXXX

SEQ ID NO: 3 - protein, 9 aa, synthetic construct
EEQFNSTYR

SEQ ID NO: 4 - protein, 49 aa, synthetic construct
DYFPEPVTVSWNSGALXXXXXXXXXXXXXXXXXXXXXXXXXXXXXXXXX

(19) United States
(12) Patent Application Publication
(10) Pub. No.: US 2026/0016485 A1
(43) Pub. Date: Jan. 15, 2026

(54) ENDOGLYCOSIDASE-ASSISTED PEPTIDE MAPPING

(71) Applicant: Regeneron Pharmaceuticals, Inc., Tarrytown, NY (US)

(72) Inventors: Haibo Qiu, Milwood, NY (US); Jieqiang Zhong, White Plains, NY (US); Ming Huang, Township of Washington, NJ (US); Ning Li, New Canaan, CT (US)

(21) Appl. No.: 19/266,789

(22) Filed: Jul. 11, 2025

Related U.S. Application Data
(60) Provisional application No. 63/670,077, filed on Jul. 11, 2024.

(51) Int. Cl. G01N 33/68

(52) U.S. Cl. CPC G01N 33/6848; G01N 2333/924; G01N 2440/38

(57) ABSTRACT

Endoglycosidase-assisted peptide mapping workflow systems for mass spectrometry (MS) characterization of non-consensus N-glycosylation in monoclonal antibodies (mAbs) are disclosed. The feasibility of the workflow was demonstrated by an atypical glycosite located within an NPNNXN (SEQ ID NO: 1) sequence in a 25-residue tryptic peptide. With the aids of endoglycosidase treatment, the resulting truncated glycan structures improved peptide ionization efficiency in MS and hence facilitated reliable quantitation of glycosite occupancy. The remaining mono-/di-saccharides served as a large mass tag allowing differentiation between the glycopeptide and deamidated peptide, thus allowing for database searching for glycosite localization and automation of the data processing workflow. This workflow offers an efficient solution for characterizing non-consensus N-glycosylation for the development of therapeutic mAbs.

This application claims priority to U.S. Application Ser. No. 63/670,077, filed Jul. 11, 2024, which is incorporated by reference in its entirety.

### FIELD OF THE INVENTIONS

The present inventions provide endoglycosidase-assisted peptide mapping workflows for mass spectrometry (MS) identification and characterization of non-consensus N-glycosylation in therapeutic polypeptides.

### REFERENCE TO ELECTRONIC SEQUENCE LISTING

This application contains a Sequence Listing which has been submitted electronically in .XML format and is hereby incorporated by reference in its entirety. Said .XML copy, created on Jul. 11, 2025, is named “135975-78502.xml” and is 4,409 bytes in size. The sequence listing contained in this .XML file is part of the specification and is hereby incorporated by reference herein in its entirety.

### BACKGROUND OF THE INVENTIONS

Glycosylation is a commonly observed protein post-translational modifications (PTMs), and plays critical roles in various biological functions, such as cell-cell signaling, protein structure modulation, and antibody immune responses.

IgG is a well-known antibody with N-glycosylation at Asn297 in the CH2 domain, a modification also known as Fc-glycosylation. It has been demonstrated that Fc-glycosylation can significantly affect the physicochemical and biological properties of antibodies, including half-life, stability, conformation, aggregation, and effector functions. In addition, it has been reported that the glycan profiles of serum antibodies differ between healthy individuals and those with rheumatic diseases. Therefore, extensive efforts have been dedicated to Fc-glycan engineering to achieve antibodies with desired properties (Raymond et al. Production of alpha-2,6-sialylated IgG1 in CHO cells. MAbs 2015; 7(3):571-583).

N-glycosylation is commonly observed on asparagine residues with the consensus motif NXS/T, in which the X at the +1 position can be any amino acid residue except for proline. This consensus can be applied to most protein N-linked glycosylation, including Fc-glycosylation of IgG. However, with the advancement of instrumentation and bioinformatics tools, an increasing number of non-consensus N-glycosylation sites have been identified. (Valliere-Douglass et al. Asparagine-linked oligosaccharides present on a non-consensus amino acid sequence in the CH1 domain of human antibodies. J Biol Chem 2009; 284(47):32493-32506; Cogez et al. N-Glycan on the Non-Consensus N-X-C Glycosylation Site Impacts Activity, Stability, and Localization of the Sd(a) Synthase B4GALNT2. Int J Mol Sci 2023; 24(4)). A commonly observed non-consensus motif is NXC. The hydrogen on the free thiol side chain of cysteine has been hypothesized to form a hydrogen bond with Asn, the glycosylation site (“glycosite”), thus facilitating the subsequent glycosylation (Imperiali et al. Asparagine-linked glycosylation: specificity and function of oligosaccharyl transferase. Bioorg Med Chem 1995; 3(12):1565-1578). More specifically, in recombinant mAbs, Valliere-Douglass et al. 2009 have identified N-glycan conjugated at Asn within an NSG sequence, which is located on the CH1 domain in the Fab region. A follow-up study from the same group discovered a novel N-glycosylation site on a glutamine residue (see Valliere-Douglass et al. Glutamine-linked and non-consensus asparagine-linked oligosaccharides present in human recombinant antibodies define novel protein glycosylation motifs. J Biol Chem 2010; 285(21):16012-16022).

The identification of non-consensus glycosites can be challenging compared to that of consensus glycosylation, which can be due to the unpredictable sequence (a sequence of three amino acids starting with the N-glycosylation site). One approach involves application of ¹⁸O water during the deglycosylation, wherein the incorporation of a heavy oxygen at the glycosite results in a +2.94 Da mass shift. However, the isotopic impurities of ¹⁸O, the back-exchange issues occurring at the peptide C-termini, and the inevitable overlapping isotopic envelopes can complicate data interpretation. Alternatively, partial deglycosylation using endoglycosidase F (Endo F) can simply remove the major portion of glycan, thus leaving one or two monosaccharide residues (GlcNAc or GlcNAcFuc) attached to peptides. This method has been used previously to investigate non-consensus N-glycosylation. However, due to the lack of proper bioinformatic tools, the acquired data were manually interpreted. In addition, to overcome the low abundance of non-consensus N-glycosylation, the previous methods have either adopted a customized multiple-reaction monitoring (MRM) strategy (Lowenthal et al. Identification of Novel N-Glycosylation Sites at Noncanonical Protein Consensus Motifs. J Proteome Res 2016; 15(7):2087-2101), which requires a survey of full MS run and the prior knowledge of the target sequon of interest (NXC, for example), or required additional enrichment followed by MS analysis. Additionally, glycosylation occupancy was not assessed or specified in the studies described above.

### SUMMARY OF THE INVENTIONS

The inventions provide systems for endoglycosidase-assisted peptide mapping workflow for a protein comprising non-consensus N-glycosylation, wherein the system can comprise the steps of: (a) providing a glycoprotein containing one or more non-consensus N-glycosites; (b) digesting the glycoprotein using trypsin; (c) de-glycosylating the digested glycoprotein by treating with an endoglycosidase, such as endoglycosidase-F2 (Endo-F2); (d) deactivating the endoglycosidase by acidifying with tri-fluoro acetic acid (TFA); (e) desalting the deactivated endoglycosidase treated tryptic digest sample, preferably by C18 pipette tips; (f) analyzing the desalted samples by nano-flow LC-MS/MS, thereby generating reduced peptide mapping data; (g) determining post-translation modifications including oxidation, Asparagine (Asn, N), deamidation, and Asp dehydration; (h) identifying glycopeptides in the sample by setting GlcNAc and GlcNAc+Fuc as common variables occur at Asn and Fc-glycosylation site; (i) validating the peptide mapping data by Extracted Ion Chromatograms (EICs) and MS spectra; and (j) identifying the non-consensus N-glycosylation and quantifying the glycosylation occupancy. The identification of the non-consensus N-glycosylation and quantification the glycosylation occupancy can be performed by processing the data obtained from the above-described steps. The data processing can perform: (i) rapid screening of glycosylated Asn and glycosite locations, (ii) the analysis of peptide mapping data, and (iii) determining the post-translation modifications. Further, the data processing can be conducted by a computer, cloud computing, artificial intelligence (AI), and/or the like.

The non-consensus N-glycosite can be glycosylated with a glycan selected from the group consisting of G0F, G1F, G2F, G2FS, G2FS1, and G2FS2. The non-consensus N-glycosite can be located at a NPNNXN (SEQ ID NO: 1) sequence in a 25-residue long tryptic peptide, wherein X can be any amino acid. The endoglycosidase-assisted peptide mapping workflow can be sensitive with a lowest detectable glycosylation occupancy of about 0.2%, which can be readily detected. The glycosylation occupancy can be calculated as:

$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 100 \%$$

The protein can be selected from the group consisting of an antibody, antibody derivative, antibody fragment, a monoclonal antibody, an Fc-containing protein, and an Fc-fusion protein, for example.

The inventions also provide methods of identifying non-consensus N-glycosylation and quantifying glycosylation occupancy in a protein, wherein the methods comprises the steps of: (a) providing a glycoprotein containing one or more non-consensus N-glycosites; (b) digesting the glycoprotein using trypsin; (c) de-glycosylating the digested glycoprotein by treating with an endoglycosidase, such as endoglycosidase-F2 (Endo-F2); (d) deactivating the endoglycosidase by acidifying with TFA; (e) desalting the deactivated endoglycosidase treated tryptic digest, preferably by C18 pipette tips; (f) analyzing the desalted samples by nano-flow LC-MS/MS, thereby generating reduced peptide mapping data; (g) determining post-translation modifications including oxidation, Asparagine (Asn, N), deamidation, and Asp dehydration; (h) identifying glycopeptides in the sample by setting GlcNAc and GlcNAc+Fuc as common variables at Asn and Fc-glycosylation site; (i) validating the peptide mapping data by Extracted Ion Chromatograms (EICs) and MS spectra; and (j) identifying the non-consensus N-glycosylation and quantifying the glycosylation occupancy. The identification of the non-consensus N-glycosylation and quantification the glycosylation occupancy can be performed by processing the data obtained from the above-described steps. The data processing can perform: (i) rapid screening of glycosylated Asn and glycosite locations, (ii) analysis of peptide mapping data, and (iii) determination of post-translation modifications. Further, data processing can be conducted by a computer, cloud computing, artificial intelligence (AI), and/or the like.

Data processing can be conducted by a computer, cloud computing, artificial intelligence (AI), and/or the like. The non-consensus N-glycosite can be glycosylated with a glycan selected from the group consisting of G0F, G1F, G2F, G2FS, G2FS1, and G2FS2. The non-consensus N-glycosite can be located at a NPNNXN (SEQ ID NO: 1) sequence in a 25-residue long tryptic peptide, wherein X can be any amino acid. The endoglycosidase-assisted peptide mapping workflow can be sensitive with a lowest detectable glycosylation occupancy of as low as about 0.2%, which can be readily detected. The glycosylation occupancy can be calculated as:

$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 100 \%$$

The protein can be selected from the group consisting of an antibody, antibody derivative, antibody fragment, a monoclonal antibody, a monospecific antibody, a bispecific antibody, an Fc-containing protein, and an Fc-fusion protein.

As disclosed herein, an enrichment strategy is not required for the characterization of non-consensus N-glycosylation using the developed workflow. A data-dependent acquisition mode with regular high-collision dissociation can be used to detect the low-abundant non-consensus glycopeptide. The application of bioinformatic tools can simplify data processing and reduce analysis time.

### BRIEF DESCRIPTION OF THE FIGURES

FIG. 1 schematically depicts substrates and products of Endo H, Endo F2 and Endo F3, individually, along with a key of common schematic representations of monosaccharides. Endo H, Endo F2, and Endo F3 can cleave oligosaccharides with high mannose structure, biantennary structure with or without core fucose, and core-fucosylated biantennary structure as well as triantennary structure, respectively. Common schematic representations of various monosaccharides as shown are used in the experiments disclosed herein. The key can apply to subsequent figures.

FIG. 2 schematically depicts deconvoluted intact Mass Spectrometry (MS) profile of mAb1. Species with higher mass (150238 Da, 150395 Da, and 153537 Da) are identified as potential uncommon glycosylated mAb according to the mass difference with respect to the regular mAb with different Fc-glycoforms on both HCs (G0F/G0F; mAb1 G0F/G1F; and mAb1 G1F/G1F). Non-glycosylated mAb1, mAb1 with Fc-glycosylation on one HC, and mAb1 with Fc-glycosylation on two HCs are annotated individually.

FIGS. 3A-3C schematically depict the results of subunit nSEC-UV/MS (native size-exclusion chromatography-ultra-violet/mass spectrometry) of mAb1, including the (3A) Total Ion Chromatogram (TIC), (3B) the UV profile at the wavelength of 280 nm (the table below demonstrates the relative abundance of peak at 6.5 min and peak at 7.2 min in the UV chromatogram), and (3C) deconvoluted mass profile of the pre-peak eluted at 6.5 min.

FIGS. 4A-4C schematically depict EICs of peptides/glycopeptides PEP: without glycosylation (FIG. 4A), with G2FS (FIG. 4B), and with G2FS2 (FIG. 4C). The occupancy of each glycoform is annotated individually in FIG. 4A and FIG. 4B.

FIGS. 5A-5B schematically depict MS2 spectra of target glycopeptide XXXXXXXXXXXXXNPNNXNXXXXXX (SEQ ID NO: 2)+G2FS without Endo F2 treatment (FIG. 5A), and glycopeptide XXXXXXXXXXXXXNPNNXNXXXXXX (SEQ ID NO: 2)+GlcNAcFuc with Endo F2 treatment (FIG. 5B).

FIGS. 6A-6B schematically depict evaluation of Endo F2 digestion efficiency using the most abundant Fc-glycopeptide EEQFNSTYR (SEQ ID NO: 3)+G0F. EICs of the target glycopeptide (precursor: [M+3H]3+, m/z: 873.3552) without Endo F2 treatment (FIG. 6A), and with Endo F2 treatment (FIG. 6B) are shown. The peak appearing in FIG. 6B at 22.75 min (labeled with an X) is the interference that has similar m/z value but with different charge state.

FIG. 7 schematically depicts a facile endoglycosidase-assisted peptide mapping workflow for the MS-based characterization of non-consensus N-glycosylation in therapeutic mAbs. Briefly, mAb samples were subjected to conventional tryptic digestion, followed by Endo-F2 digestion. TFA (tri-fluoro acetic acid) was then added to the resulting digests to deactivate the enzymes before desalting with C18 pipette tips (can be referred to as C18 Toptips). Subsequently, the purified samples were subjected to nanoLC-MS/MS, and the acquired data were processed for initial identification, and manual validation, respectively.

FIGS. 8A-8B schematically depict the MS2 spectra of the target glycopeptides generated by BYONIC™. The BYONIC™ filtered matches for the non-consensus glycopeptides with two glycoforms after Endo F2 treatment, including PEP+GlcNAcFuc (FIG. 8A), and PEP+GlcNAc. Both matches have scores higher than 900 (FIG. 8B). Fragments of b ions, fragments of y ions, and fragments of other ions, such as oxonium ions are identified in FIGS. 8A and 8B. Specifically in FIG. 8B, y ions annotated as “˜y” are the y ions without GlcNAc.

FIGS. 9A-9C schematically depict MS2 (Tandem Mass Spectrometry, MS/MS) spectrum (m/z ranging from 120 to 1700) of the targeted glycopeptide PEP+GlcNAcFuc after Endo F2 treatment (FIG. 9A). Zoom-in spectra for m/z range 120-700, and m/z range 750-1700, are displayed as shown in FIG. 9B and FIG. 9C, respectively. Oxonium ions including GlcNAc-C₂H₆O₃ with m/z 126, GlcNAc-CH₆O₃ with m/z 138, Fuc with m/z 147, GlcNAc-2H₂O with m/z 168, and GlcNAc with m/z 204 are annotated in FIG. 9B as —C₂H₆O₃, —CH₆O₃, triangles, —2H₂O, and squares, respectively.

FIGS. 10A to 10C schematically depict N-glycosylation site mapping with sequential enzymatic treatments using trypsin, PNGase F, and Asp-N. The deamidation resulted from the deglycosylation prompted the cleavage at the N-terminal of the glycosylation site using Asp-N, and therefore the original PEP was cleaved and resulted in peptide DXNXXXXXX. FIG. 10A is the EIC of peptide DXNXXXXXX in the negative control sample using trypsin and Asp-N. FIG. 10B is the EIC of peptide DXNXXXXXX in the sample using trypsin, PNGase F, and Asp-N, and FIG. 10C is the corresponding MS/MS spectrum.

FIG. 11 schematically depicts a predicted structure and local environment of the NPNNXN (SEQ ID NO: 1) region on mAb1 generated by Molecular Operating Environment (MOE), with the four asparagine (Asn) residues highlighted using arbitrary numbering (AsnI, Asn3, Asn4, and Asn6). The Asn backbone oxygen atoms and Asn side chain nitrogen atoms are identified according to the key. The carboxyl oxygen atoms from the amide bonds of Asn residues, carbon atoms, hydrogen atoms, and predicted hydrogen bonds between carboxyl oxygens and N-linked hydrogen atoms are as indicated in FIG. 11. Residue Solvent Accessible Surface Area (SASA) was calculated in MOE.

FIGS. 12A-12B schematically depict Extracted Ion Chromatograms (EICs) of glycopeptides PEP+GlcNAc/GlcNAcFuc with Endo F2 treatment (no separation was observed with the two glycopeptide species) and non-glycosylated peptide PEP (FIG. 12A) and quantitation of the glycosylation occupancy on Asn 4 (FIG. 12B).

FIGS. 13A-13B schematically depict the results of post-digestion stability evaluation of samples treated with Endo F2 over 30 days. EICs of unglycosylated peptide PEP and glycopeptides PEP+GlcNAc/GlcNAcFuc of data acquired on day 1 (FIG. 13A) and day 30 (FIG. 13B) are illustrated. Samples were stored in an auto-sampler set at 4° C. for 30 days between the runs.

FIGS. 14A-14F schematically depict EICs of the DYFPEPVTVSWNSGALXXXXXXXXXXXXXXXXXXXXXXXXXXXXXXXXX (SEQ ID NO: 4) tryptic peptides with or without glycosylation on mAb2 (FIGS. 14A, 14B, and 14C) and mAb3 (FIGS. 14D, 14E, and 14F). Both products after Endo F2 digestion, peptide+GlcNAc, and peptide+GlcNAcFuc were identified. The glycosylation occupancies of the shown peptides were 0.2% and 0.3% for mAb2 and mAb3, respectively.

### DETAILED DESCRIPTION OF THE INVENTIONS

Unless stated otherwise, all technical and scientific terms and phrases used herein have the same meaning as commonly understood by one of ordinary skill in the art to which the invention belongs.

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Protein,” “Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

The term “sequon” of a polypeptide refers to a polypeptide variant that includes in its amino acid sequence an “exogenous N-linked glycosylation sequence.” A “sequon” polypeptide contains at least one exogenous N-linked glycosylation sequence, but may also include one or more endogenous (e.g., naturally occurring) N-linked glycosylation sequence.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. (Butler and Spearman, “The choice of mammalian cell host and possibilities for glycosylation engineering”, Curr. Opin. Biotech. 30:107-112 (2014)).

Antibodies are often used as therapeutic biomolecules. An antibody can include immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” can include an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” can include an immunoglobulin heavy chain constant region sequence from any organism, and unless otherwise specified can include a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain can include a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” can include an immunoglobulin light chain constant region sequence from any organism, and unless otherwise specified can include human kappa and lambda light chains. Light chain variable (VL) domains typically include three light chain CDRs and four framework (FR) regions, unless otherwise specified. Generally, a full-length light chain can include, from amino terminus to carboxyl terminus, a VL domain that can include FR1-CDR1-FR2-CDR2-FR3-CDR3-FR4, and a light chain constant domain. Light chains that can be used with these inventions include those, for example, which do not selectively bind either the first or second antigen selectively bound by the antigen-binding protein. Suitable light chains include those that can be identified by screening for commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” can include an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” can include an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region” (“CDR”) can include an amino acid sequence encoded by a nucleic acid sequence of an organism's immunoglobulin genes that normally (that is, in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include but are not limited to: antibody fragments (for example, ScFv-Fc, dAB-Fc, half antibodies, Fab), multispecifics (for example, IgG-ScFv, IgG-dab, ScFV-Fc-ScFV, tri-specific). “Fab” refers to an antibody fragment comprising an antigen binding region. A Fab typically will lack the Fc portion.

The phrase “Fc-containing protein” can include antibodies, bispecific antibodies, antibody derivatives containing an Fc, antibody fragments containing an Fc, Fc-fusion proteins, receptor Fc-fusion proteins (including trap proteins), immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor, and also unable to activate complement, then the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins, such as antibodies, can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V2591), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

“Fv” stands for fragment variable and is primarily responsible for binding to epitopes.

As used herein, the expression “formulation” means a combination of at least one active ingredient (e.g., a protein, such as polypeptide, antibody, monoclonal antibody, etc. which is capable of exerting a biological effect in a human or non-human animal), and at least one inactive ingredient which, when combined with the active ingredient or one or more additional inactive ingredients, is suitable for therapeutic administration to a human or non-human animal. The term “formulation,” as used herein, means “pharmaceutical” or “therapeutic” formulation unless specifically indicated otherwise. The present invention provides pharmaceutical formulations comprising at least one therapeutic protein. According to the present invention, the therapeutic protein is an antibody, or an antigen-binding fragment thereof. The term “formulation,” as used herein, also means a solid, semi-solid, and/or liquid formulation, such as suitable for oral, intramuscular, subcutaneous, and/or intravenous administration.

An “Endoglycosidase” is a type of glycan-specific glycan-cleaving enzyme. The term covers any endoglycosidase enzyme, including Endo-β-N-acetylglucosaminidase (Endo-NAG), Endo F1, Endo F2, Endo F3, Endo A, Endo D, Endo H, Endo M, and Endo S.

Various methods of stability testing are known in the art, including Fleischman et al. Shipping-Induced Aggregation in Therapeutic Antibodies: Utilization of a Scale-Down Model to Assess Degradation in Monoclonal Antibodies. J. Pharm. Sci. (2017) 106: 994-1000; Ghazvini et al. Evaluating the Role of the Air-Solution Interface on the Mechanism of Subvisible Particle Formation Caused by Mechanical Agitation for an IgG1 mAb1. J. Pharm. Sci. (2016) 105: 1643-1656; and Torisu et al. Synergistic Effect of Cavitation and Agitation on Protein Aggregation. J. Pharm. Sci. (2017) 106: 521-529).

As used in this specification and the appended claims, the singular forms “a,” “an,” and “the” include plural references unless the context clearly dictates otherwise. Thus, for example, a reference to “a method” can include one or more methods, and/or steps of the type described herein and/or which will become apparent to those persons skilled in the art upon reading this disclosure and so forth.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### DESCRIPTION

N-linked glycosylation, an extensively studied protein post-translational modification, was conventionally understood to occur at asparagine (Asn or N) sites with the consensus motif NXS/T, where X can be any amino acid residue except for proline, followed by serine or threonine. However, with advancements in characterization techniques and bioinformatic tools, increasing evidence indicates that Asn residues that are not located in the NXS/T consensus motif can also undergo N-glycosylation, which is also known as non-consensus or noncanonical N-glycosylation. Yet, it remains challenging to characterize the non-consensus N-glycosylation because of the unpredictable sequence (such as NPNNXN (SEQ ID NO: 1) sequence, where X can be any amino acid) and its relatively low abundance.

The characterization of non-consensus N-glycosylation can be important for: (i) determining the glycosite, (ii) the relative abundance of the glycosite, and (iii) the nature of the glycoforms. The identification of non-consensus glycan site can be even more challenging compared to that of consensus glycan, which can be mainly due to the unpredictable sequence. One approach can be to apply ¹⁸O water during the deglycosylation, incorporating a heavy oxygen at the glycosite, which can subsequently result in a +2.94 Da mass shift.

Depending on the location and the abundance of non-consensus glycosylation occurring on mAb, the molecule might be impacted to different degrees. It is known that glycosylation on complementarity-determining region (CDR) may affect the antibody-antigen binding. Therefore, it is critical to localize and quantify the unpredictable non-consensus glycosylation in the early development stage of mAb.

The current inventions present an improved method that was demonstrated in a challenging case study. A suspicious non-consensus N-glycosylation was discovered and identified on the Fab region with the regular intact mass analysis and subunit analysis. To precisely localize the glycosite, an Endo F-assisted peptide mapping is developed with application of assays incorporating bioinformatic software.

Intact MS analysis of a protein sample is performed to determine whether non-consensus glycosylation is present, thereby determining the potential form of the glycosylation based on mass difference compared to the main species. The inventions provide systems for endoglycosidase-assisted peptide mapping workflow for the proteins that comprise non-consensus N-glycosylation, wherein the system can comprise the steps of: (a) providing the glycoprotein containing one or more non-consensus N-glycosites; (b) digesting the glycoprotein using trypsin; (c) de-glycosylating the digested glycoprotein by treating with an endoglycosidase, such as Endo-F2; (d) deactivating the endoglycosidase by acidifying with TFA; (e) desalting the deactivated endoglycosidase treated tryptic digest sample by C18 pipette tips; (f) analyzing the desalted samples by nano-flow LC-MS/MS, thereby generating reduced peptide mapping data; (g) determining post-translation modifications including oxidation, Asparagine (Asn, N), deamidation, and Asp dehydration; (h) identifying glycopeptides in the sample by setting GlcNAc and GlcNAc+Fuc as common variables occur at Asn and Fc-glycosylation site; (i) validating the peptide mapping data by Extracted Ion Chromatograms (EICs) and MS spectra; (j) identifying the non-consensus N-glycosylation and quantifying the glycosylation occupancy by data processing that is utilized for: (i) rapid screening of glycosylated Asn and glycosite locations, (ii) analyzing peptide mapping data, and (iii) determining the post-translation modifications.

The selection of the endoglycosidase can be case-dependent, and the type of the enzyme can be adjustable accordingly. According to the manufacturer (New England Biolabs) Endo-F2 is a highly specific recombinant endoglycosidase which cleaves within the chitobiose core of asparagine-linked complex biantennary and high mannose oligosaccharides from glycoproteins and glycopeptides. Endo-F2 cleaves biantennary glycans at a rate approximately 20 times greater than high mannose glycans. The activity of Endo-F2 is identical on biantennary structures with and without core fucosylation. However, Endo-F2 is not active on hybrid or tri- and tetra-antennary oligosaccharides.

The non-consensus N-glycosite can be glycosylated with a glycan selected from the group consisting of G0F, G1F, G2F, G2FS, G2FS1, and G2FS2. The non-consensus N-glycosite can be located at a NPNNXN (SEQ ID NO: 1) sequence in a 25-residue long tryptic peptide, wherein X can be any amino acid. The endoglycosidase-assisted peptide mapping workflow developed according to the current inventions enable the detection of non-consensus N-glycosylation, which is not detectable with conventional peptide mapping workflow.

The endoglycosidase-assisted peptide mapping workflow also can be sensitive with a lowest detectable glycosylation occupancy of about 0.2%, which can be readily detected. The glycosylation occupancy can be calculated as:

$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 100 \%$$

Briefly, the samples are tryptic digested, which are then treated with endoglycosidase (for example, Endo F 2) for the glycan truncation. The resulting peptides and glycopeptides without additional enrichment are subsequently injected to liquid chromatography-mass spectrometry (LC-MS) using the regular data-dependent acquisition (DDA) mode. Later, the data are processed by software analysis for the automated screening. The potential glycosite is confirmed to be located at a region that has 4 Asn within 6 residues (NPNNXN) (SEQ ID NO: 1) on a 25-residue long tryptic peptide. Furthermore, the automated data interpretation can be assessed. The glycosylation occupancy can be accurately quantified, minimizing the bias introduced by the glycan moiety. To demonstrate the feasibility of the developed assay, the methods of the instant inventions have been successfully applied to other mAbs.

The inventions are amenable to use with a wide variety of Fc-containing proteins and other proteins. The inventions can be employed in the production of biological and pharmaceutical products. For example, for antibodies, the inventions are amendable for research and production use for diagnostics and therapeutics based upon all major antibody classes, namely IgG, IgA, IgM, IgD and IgE. IgG is a preferred class, such as IgG1 (including IgG1k and IgG1κ), IgG2 and IgG4. Exemplary antibodies to be analyzed and produced according to the inventions include, but are not limited to, Alirocumab, Atoltivimab, Maftivimab, Odesivimab, Odesivivmab-ebgn, Casirivimab, Imdevimab, Cemiplimab, Cemplimab-rwlc, Dupilumab, Evinacumab, Evinacumab-dgnb, Fasimumab, Nesvacumab, Trevogrumab, Rinucumab and Sarilumab. Antibodies can include a human antibody, a humanized antibody, a chimeric antibody, a monoclonal antibody, a monospecific antibody, a multispecific antibody, a bispecific antibody, an antigen binding antibody fragment, a single chain antibody, a minibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. The antibody can be an IgG1 antibody. The antibody can be an IgG2 antibody. The antibody can be an IgG3 antibody. The antibody can be an IgG4 antibody. The antibody can be a chimeric IgG2/IgG4 antibody. The antibody can be a chimeric IgG2/IgG1 antibody. The antibody can be a chimeric IgG2/IgG1/IgG4 antibody.

In addition to the antibodies described in the Examples and Figures, the antibody can be selected from the group consisting of an anti-Programmed Cell Death 1 antibody (e.g., an anti-PD1 antibody as described in U.S. Pat. Appln. Pub. No. US2015/0203579A1), an anti-Programmed Cell Death Ligand-1 (e.g., an anti-PD-L1 antibody as described in in U.S. Pat. Appln. Pub. No. US2015/0203580A1), an anti-Dll4 antibody, an anti-Angiopoetin-2 antibody (e.g. an anti-ANG2 antibody as described in U.S. Pat. No. 9,402,898), an anti-Angiopoetin-Like 3 antibody (e.g. an antiAngPtl3 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an 25 anti-C5 antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g., an anti-PCSK9 antibody as described in U.S. Pat. No. 8,062,640 or U.S. Pat. Appln. Pub. No. US2014/0044730A1), an anti-Growth and Differentiation Factor-8 antibody (e.g., an anti-GDF8 antibody, also known as anti-myostatin antibody, as described in U.S. Pat. Nos. 8,871,209 or 9,260,515), an anti-Glucagon Receptor (e.g., anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g an antiIL4R antibody as described in U.S. Pat. Appln. Pub. No. US2014/0271681A1 or U.S. Pat. Nos. 8,735,095 or 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. Appln. Pub. Nos. US2014/0271658A1 or US2014/0271642A1), an anti-Respiratory syncytial virus antibody (e.g., anti-RSV antibody as described in U.S. Pat. Appln. Pub. No. US2014/0271653A1), an anti-Cluster of differentiation 3 (e.g., an anti-CD3 antibody, as described in U.S. Pat. Appln. Pub. Nos. US2014/0088295A1 and US20150266966A1, and in U.S. Application No. 62/222,605), an anti-Cluster of differentiation 20 (e.g., an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation 48 (e.g., anti-CD 48 antibody as described in U.S. Pat. No. 9,228,014), an anti-Fel d1 antibody (e.g., as described in U.S. Pat. No. 9,079,948), an anti-Middle East Respiratory Syndrome virus (e.g., an anti-MERS antibody as described in U.S. Pat. Appln. Pub. No. US2015/0337029A1), an anti-Ebola virus antibody (e.g., as described in U.S. Pat. Appln. Pub. No. US2016/0215040), an anti-Zika virus antibody, an anti-Lymphocyte Activation Gene 3 antibody (e.g., an anti-LAG3 antibody, or an anti-CD223 antibody), an anti-Nerve Growth Factor antibody (e.g., an anti-NGF antibody as described in U.S. Pat. Appln. Pub. No. US2016/0017029 and U.S. Pat. Nos. 8,309,088 and 9,353,176) and an anti-Activin A antibody. The bispecific antibody can be selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (e.g., an anti-CD3×anti-Muc16 bispecific antibody), and an anti-CD3×anti-Prostate-specific membrane antigen bispecific antibody (e.g., an anti-CD3×anti-PSMA bispecific antibody). See also U.S. Patent Publication No. US 2019/0285580 A1.

The inventions also are amenable to the production of other molecules, including fusion proteins. Preferred fusion proteins include Receptor-Fc-fusion proteins, such as Trap proteins. the protein of interest is a recombinant protein that contains an Fc moiety and another domain, (e.g., an Fc-fusion protein). The Fc-fusion protein can be a receptor Fc-fusion protein, which contains one or more extracellular domain(s) of a receptor coupled to an Fc moiety. The Fc moiety can comprise a hinge region followed by a CH2 and CH3 domain of an IgG. The receptor Fc-fusion protein can contain two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the Il-1R1 extracellular region fused to Fc of hIgG1; see U.S. Pat. No. 6,927,044, or a VEGF trap (e.g., aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). The Fc-fusion protein can be a ScFv-Fc-fusion protein, which contains one or more of one or more antigen binding domain(s), such as a variable heavy chain fragment and a variable light chain fragment, of an antibody coupled to an Fc moiety.

Other proteins lacking Fc portions, such as recombinantly produced enzymes and mini-traps, also can be made according to the inventions. Mini-traps are trap proteins that use a multimerizing component (MC) instead of an Fc portion and are disclosed in U.S. Pat. Nos. 7,279,159 and 7,087,411. Derivatives, components, domains, chains, and fragments of the above also are included.

The inventions are further described by the following examples, which do not limit the inventions in any manner. The order of performance of the below experiments and/or examples or example steps can be altered or combined as determined by the person of skill in the art as informed by and in view of the teachings and data contained herein.

### Examples

### Experimental Approaches:

### Materials:

All mAbs used below were produced in-house at Regeneron Pharmaceuticals (Tarrytown, NY). Acetonitrile (ACN, LC-MS grade), trifluoracetic acid (TFA), formic acid (FA), iodoacetamide (IAA), tris-(2-carboxyethyl) phosphine hydrochloride (TCEP-HCl), Tris-HCl buffer (pH 7.5), and C18 pipette tips were purchased from Thermo Fisher Scientific (Waltham, MA). Urea was purchased from Sigma Aldrich (St. Louis, MO). Sequencing grade-modified trypsin was purchased from Promega (Madison, WI). PNGase F and Endo F2 were purchased from New England Biolabs (Ipswich, MA). FabRICATOR was purchased from Genovis (Cambridge, MA). Deionized water was acquired from a Milli-Q integral water purification system with a MilliPak Express 20 filter (MilliporeSigma, Burlington, MA).

### LC-MS Intact Mass Analysis:

The mAb sample was diluted with deionized water and injected into a Waters Xevo G3 QTOF Mass Spectrometry system equipped with a Waters ACQUITY UPLC system (Milford, MA) for intact mass analysis. Samples were separated on a Waters BioResolve polyphenyl column (2.7 μm, 2.1×150 mm). Mobile phase A comprised 0.1% FA in water, and mobile phase B (MPB) comprised 0.1% FA in 80% ACN. Samples were separated at 80° C. with a short ramp gradient starting from 10% MPB, which was then increased to 90% over 4 min, and maintained for 1 min. Pre-conditioning of the column for the subsequent run lasted for 2 min with 10% MPB. The flow rate of the run was fixed at 300 μL/min. The scan range of the MS operated in positive mode was set from 500 to 5000 Da with a 0.5s scan time. The source parameters were set as follows: capillary voltage, 3 kV; cone voltage. 150 V; source temperature, 115° C.; cone gas, 50 L/h; and desolvation gas, 1000 L/h.

Subunit Native Size Exclusion Chromatography (nSEC)-MS Analysis:

The method was adapted from the Yan et al. (Post-Column Denaturation-Assisted Native Size-Exclusion Chromatography-Mass Spectrometry for Rapid and In-Depth Characterization of High Molecular Weight Variants in Therapeutic Monoclonal Antibodies. J Am Soc Mass Spectrom 2021; 32(12):2885-2894). In brief, samples were deglycosylated with PNGase F diluted with 50 mM Tris-HCl, pH 7.0 at a ratio of 1 IUB milliunit per 10 μg of protein. The incubation was performed at 45° C. for 1 hour. Subsequently, FabRICATOR (1 IUB milliunit per 1 μg protein) resuspended in 50 mM Tris-HCl, pH 7.5 was added to the samples and incubated 1-hour at 37° C. to cleave the mAb into F(ab′)₂ and Fc fragments.

### Nano-flow LC-MS/MS Peptide Mapping Analysis:

The mAbs were subjected to the standard peptide mapping preparation process, including reduction, alkylation, and enzymatic digestion. Briefly, 5 mM TCEP-HCl was added to mAb samples resuspended in 5 mM acetic acid aqueous solution. The mixtures were then incubated at 80° C. for 10 min to denature and reduce the proteins. After the mixtures had cooled to room temperature, 1M urea dissolved in 1M Tris-HCl buffer, pH 7.5 was added. Subsequently, samples were alkylated and digested with 1 mM IAA and trypsin (E/S=1:20, w/w) at 37° C. for 3 hours.

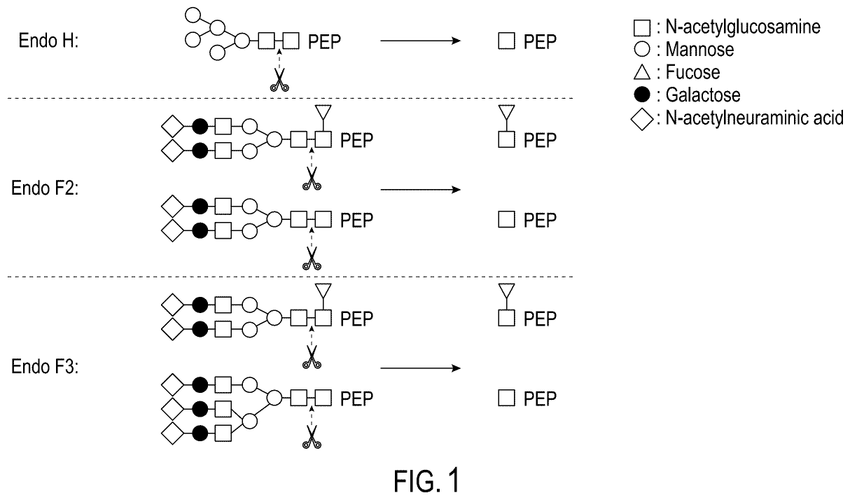

For samples subjected to partial deglycosylation with endoglycosidase (here, Endo F2), GlycoBuffer 4 was added to the resulting tryptic digests, reaching to the final pH at ˜5, and 1-hour digestion with Endo F2 (E/S=1:10, w/w) was performed at 37° C. Endo F2 was selected according to the observed potential glycoform (common endoglycosidases and the corresponding suitable substrates are schematically depicted in FIG. 1 according to the vendor's instructions). For enzyme deactivation, the digests were acidified with TFA (final concentration 1%). For details on peptide samples subjected to PNGase F digestion and Asp-N digestion, see the Supporting Information. Subsequently, C18 pipette tips were used to desalt the peptide mixtures prior to nano-flow LC-MS/MS analysis. Other desalting materials and approaches also can be used according to the inventions.

A Q-Exactive HF-X Hybrid Quadrupole-Orbitrap MS (Thermo Fisher Scientific, San Jose, CA) coupled to an Ultimate 3000 nanoLC system (Thermo Fisher Scientific, Sunnyvale, CA) was used for the bottom-up analysis. The purified peptides/glycopeptides were first loaded onto an Acclaim PepMap 100 C18 trap (0.1 mm×20 mm, 5 μm, Thermo Fisher Scientific, Sunnyvale, CA), then washed with mobile phase A (0.1% FA in H₂O) at 5 μL/min for 5 min. The valve position was then switched to connect the column (C18 integrated column, 25 cm×75 μm×1.7 μm, CoAnn Technologies, Richland, WA) and the trap. Separation was achieved with a programed gradient starting at 5% MPB (0.1% FA in 80% ACN) with a fixed flow rate of 250 nL/min. Mobile phase B was first increased to 50% over 65 min, and was subsequently increased to 95% within 10 min, which was then maintained for 10 min to completely elute all analytes. Post-equilibration with 5% mobile phase B was performed for 10 min before the next injection. Full MS was operated in positive mode with the following parameters: nanoESI voltage, 2 kV; capillary temperature, 325° C.; and scan range, 200 to 2000 m/z. The MS acquisition in data-dependent acquisition (DDA) mode consisted of a full mass scan followed by MS/MS scans of the 10 most abundant ions with the following settings: automated gain control AGC target, 1E5; isolation window, 2 m/z; and normalized collision energy, (NCE) 27. Dynamic exclusion was enabled with a window of 10 seconds.

LC-MS Settings for Subunit nSEC-MS Analysis:

An Ultimate3000 UHPLC system (Thermo Fisher Scientific, Bremen, Germany) equipped with an Acquity BEH200 SEC column (4.6×300 mm, 1.7 μm, 200 Å, Waters, Milford, MA) was used to conduct the nSEC. Samples were separated with isocratic 150 mM ammonium acetate at a fixed flow rate of 0.2 mL/min, and the column compartment was set at 30° C. MS analysis was performed on a Thermo Q Exactive UHMR (Thermo Fisher Scientific, Bremen, Germany) instrument equipped with a microflow-nanospray electrospray ionization source and a microfabricated monolithic multinozzle emitter (Newomics, Berkeley, CA). The MS parameters were set as follows: m/z range, 2000-15000; MS resolution, 12500 (UHMR) or 17500 (EMR); capillary spray voltage, 3.0 kV; S-lens RF, 200; in-source fragmentation energy, 100; and HCD trapping gas pressure, 3.

### C18 Clean-up:

The purification method was adapted from the vendor's instructions. In brief, 100 μL 50% ACN was used to wet the bead bed, and a triple wash with 0.1% TFA was performed to equilibrate the tip. Subsequently, 100 μL samples were aspirated and dispensed ten times before rinsing with 100 μL of 5% ACN with 0.1% TFA twice. Finally, samples were eluted with 100 μL 60% ACN with 0.1% TFA. The purified samples were dried in a speedVac and resuspended in MPA before LC-MS.

### PNGase F Digestion on Tryptic Peptide:

The resulted tryptic peptides were followed by PNGase F digestion for the complete deglycosylation (if needed). PNGase F was added to the samples after trypsin digestion at the ratio of 1 μL of enzyme to 50 μg of protein, and the incubation was conducted at 37° C. for one hour.

### Asp-N Digestion on Tryptic Peptide:

Asp-N was added (E/S=1:50, w/w) after PNGase F digestion or trypsin digestion (with or without deglycosylation), and samples were incubated at 37° C. for 3 hours. All peptide samples were subjected to C18 clean-up prior to LC-MS/MS analysis.

### Data Analysis:

The acquired intact MS and subunit MS data were processed and deconvoluted with PMi Intact Mass (Protein Metrics, Cupertino, CA). The parameters for the deconvolution were as follows: mass range, 143000-163000 (intact), 30000-110000 (subunit); m/z range (mass spectrometry data mass-to-charge ratio, m/z, where m is the molecular weight of the ion (in daltons) and z is the number of charges present on the measured molecule), 1000-4000 (intact), 4000-6500 (subunit); charge vector spacing, 0.2; baseline radius, 2; and charge range, 5-100. Default settings were used for the other parameters.

BYONIC™ (Protein Metrics, Cupertino, CA) was used to search the acquired reduced peptide mapping data. Common post-translational modifications, including Met/Trp oxidation, Asn deamidation, and Asp dehydration, were set as common variables. For glycopeptide identification, GlcNAc (+203.0794 Da) and GlcNAc+Fuc (+349.1373 Da) were set as common variable modifications occurring at Asn, in addition to the regular Fc-glycosylation. The generated results were imported into BYOLOGIC™ (Protein Metrics, Cupertino, CA) with a score threshold set at 500 for assessment, for example, checking the corresponding EICs, full MS spectra, and MS/MS spectra to validate the software assignment. All extracted ion chromatograms (EICs) and MS spectra were generated with Skyline (MacCoss Lab Software, Seattle, WA) and/or Xcalibur QualBrowser (Thermo Fisher Scientific, San Jose, CA) with a mass tolerance of 10 ppm.

### In-Silico 3D Homology Modeling:

To understand the local environment of the mAb1 Fab region, a homology model was built by constructing appropriate assembly of HC and LC according to IgG model protocols in Molecular Operating Environment (MOE) (2022.02, Chemical Computing Group ULC, Montreal, Canada) with the Antibody Modeler application. A homology search was performed for each sequence to identify optimal templates. The templates were then selected automatically from the built-in database, and the resulting antibody structures were extracted, analyzed, and exported. The residual solvent-accessible surface area (SASA) was calculated for selected Asn residues of interest under physiological conditions (pH 7.4) in the MOE package.

### Results and Analyses:

A case study of the non-consensus N-glycosylation of a mAb is disclosed, which has been difficult to characterizing due to the asparagine-rich nature of the potential glycosite-related peptide (with the sequence of NPNNXN (SEQ ID NO: 1)). To address this circumstance, an Endo F-assisted peptide mapping workflow was developed, which incorporated a software for data processing. The developed method was demonstrated to be sensitive, accurate, and reliable. The discovery of non-consensus N-glycosylation with regular MS analysis, the limitations of the conventional MS assays, the improvements gained from Endo F treatment, and the application of the developed workflow onto other mAbs are disclosed herein.

Discovery of the Non-Consensus N-Glycopeptide on mAb1:

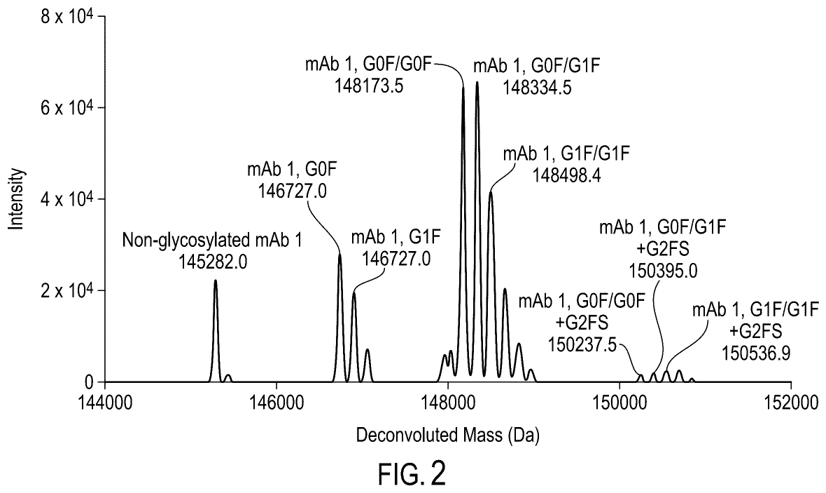

Intact mass analysis can be routinely applied strategy to monitor properties of the protein therapeutics, particularly those in early development stages, such as mAb1. As schematically depicted in FIG. 2, the deconvoluted intact mass profile of mAb1. MS peaks of the non-glycosylated mAb, mAb with Fc-glycosylation on one heavy chain, and mAb with Fc-glycosylation on both heavy chains were identified individually. In addition to the above-mentioned identifications, noticeable MS peaks in the high mass range (150237.5 Da, 150395 Da, and 150536.9 Da) were observed. The mass differences with respect to the regular mAb with Fc-glycosylation on both heavy chains (mAb1, G0F/G0F, 148173.5 Da; mAb1, G0F/G1F, 148334.5 Da; and mAb1, G1F/G1F, 148498.4 Da) were calculated to be approximately 2050 Da. Therefore, it is possible that the mass shifts resulted from additional N-linked glycosylation on mAb1. N-Glycan G2FS with an exact mass of 2059.7 Da was preliminarily considered the potential glycoform, according to the mass difference. Notably, the deconvoluted mass for the species with low abundance, such as the peaks in the high mass range as schematically depicted in FIG. 2, can suffer from relatively large mass errors due to poor signal-to-noise (S/N) ratios.

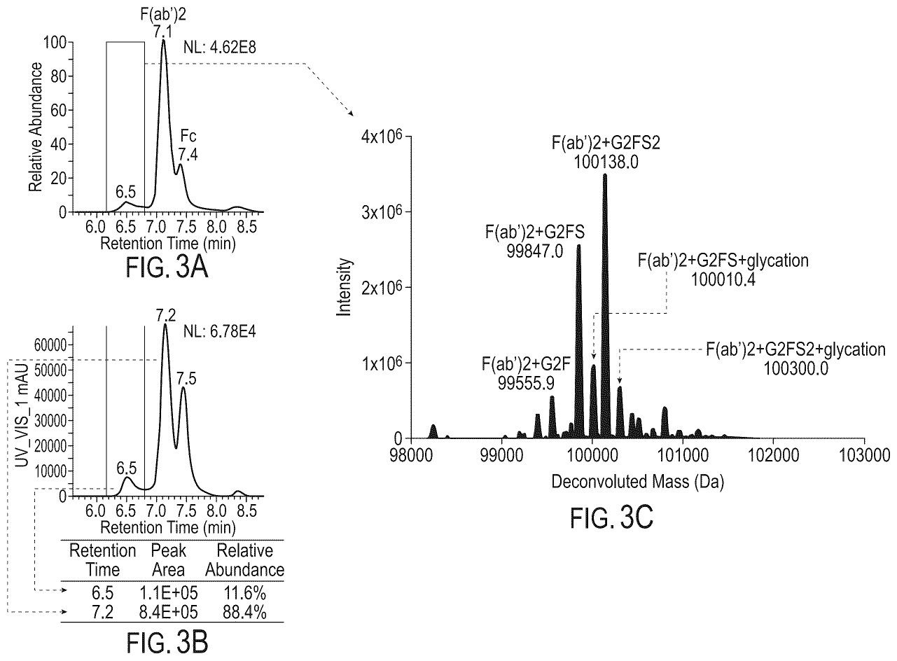

To further validate the presence of additional N-glycosylation and to map the potential modification site, subunit analysis was performed with FabRICATOR, and the results are schematically depicted in FIGS. 3A-3C. After FabRICATOR digestion, mAb1 was cleaved into 2 major species, F(ab′)₂ and Fc, which eluted at 7.1 min and 7.4 min, respectively (FIG. 3A). In addition, a pre-peak appeared at 6.5 min and was identified as a protein species with the corresponding UV absorbance profile at 280 nm (FIG. 3B). The peaks eluted at 6.5 min and 7.2 min have a relative abundance of 11.6%, and 88.4%, respectively (embedded Table under FIG. 3B). The deconvoluted mass profile of the pre-peak (FIG. 3C) indicated that multiple glycoforms, including G2F, G2FS, and G2FS2, were associated with the Fab region. Intriguingly, despite the effective removal of Fc N-glycans through PNGase F treatment, the Fab-glycosylation remained intact. These findings suggested that deglycosylation of the Fab region might not be efficient under native conditions.

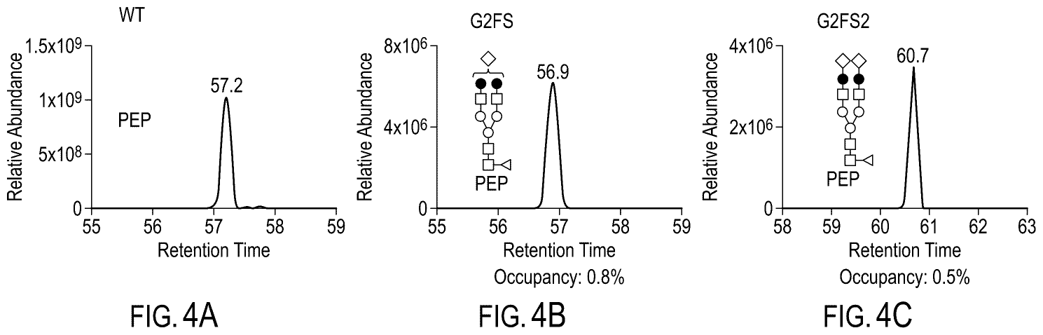

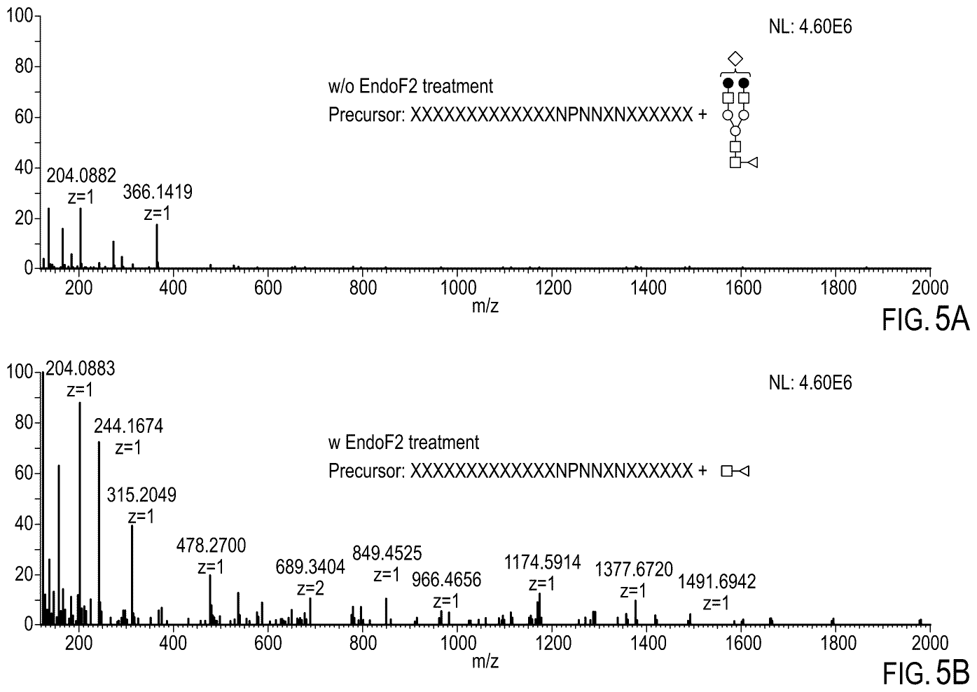

Subsequently, the protein sequence of each tryptic peptide of mAb1 was assessed. Canonical motif (NXS/T) in the Fab region was identified. Therefore, it is possible that the Fab-glycosylation on mAb1 can be a non-consensus N-linked glycosylation, thus suggesting that every asparagine in the Fab region might have been a potential glycosylation site. Among all tryptic peptides containing asparagine, the CDR peptide XXXXXXXXXXXXXNPNNXNXXXXXX (SEQ ID NO: 2) (abbreviated as PEP unless otherwise specified) was notable, because it contained four asparagine residues within a six-residue sequence, which can be more likely to be glycosylated. Subsequently, PEP+G2FS and PEP+G2FS2 were searched and extracted from the regular tryptic peptide mapping data (FIGS. 4A-4C). In the full MS profile, the two glycopeptides were identified with low relative abundance (0.8% and 0.5% as schematically depicted in FIGS. 4B and 4C, respectively). However, extremely limited peptide backbone fragments were detected in the corresponding MS2 spectrum (FIG. 5A) due to the low abundance, and these findings were insufficient to confirm whether the glycopeptide was derived from PEP and to localize the exact glycosylation site.

### Simple Endo F-Assisted Peptide Mapping Workflow to Characterize Non-Consensus N-Glycosylation:

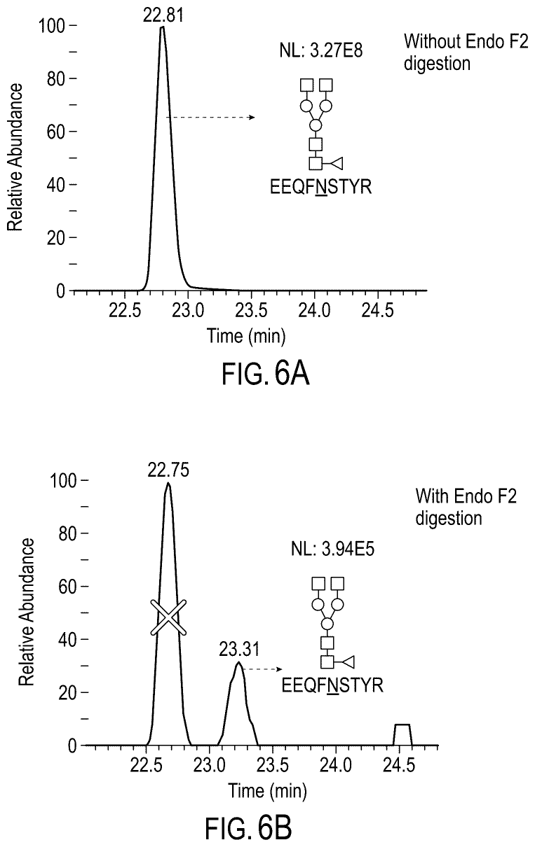

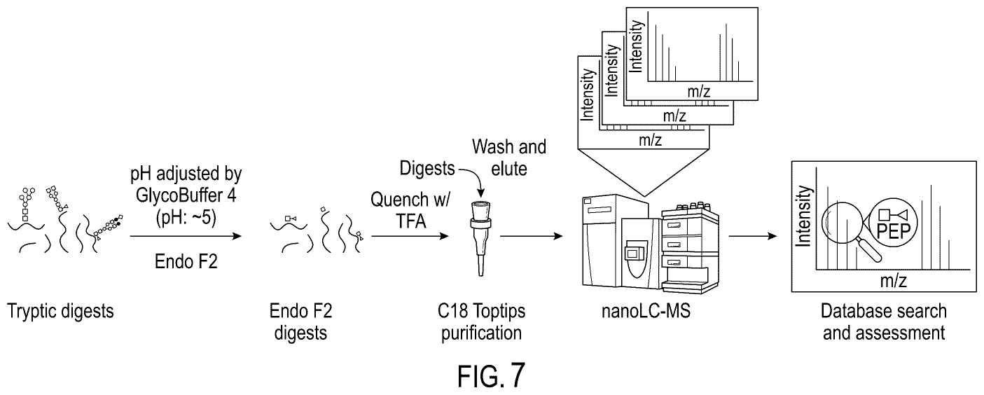

Despite the extensive characterization efforts combining intact mass analysis and conventional peptide mapping approaches described in the previous section, questions remained: 1) whether the abnormal MS peaks observed in FIG. 2 and FIGS. 3A-3C were glycopeptides, and, if so, 2) where the glycosylation occurred, and 3) how abundant the glycosylation was. To answer these questions, a bottom-up workflow was developed, as schematically depicted in FIG. 7, which used endoglycosidase to partially remove the ambiguous conjugated glycans. Combination of multiple endoglycosidases (including Endo H, Endo D, Endo F2, Endo F3, etc.) can be applied when working on the glycoproteins/glycopeptides with unknown glycoforms. Here, Endo F2 was selected based on the potential glycoform observed in our intact mass analysis (FIG. 2) and subunit analysis (FIGS. 3A-3C). The digestion efficiency of Endo F2 was evaluated with the Fc-glycosylation EEQFNSTYR (SEQ ID NO: 3)+G0F, and it shows more than 99.9% of glycan removal rate as in FIGS. 6A-6B. Additionally, in this workflow, a commercially available peptide MS/MS database searching software was used to allow for degrees of automated data processing, thus substantially decreasing the workload and the data processing time. In brief, endoglycosidase was added after the typical tryptic digestion for partial deglycosylation. Samples were then desalted with C18 tips before the nano-flow LC-MS/MS analysis. The resultant peptide mapping data were searched with BYONIC™. The filtered matches were further assessed with BYOLOGIC™.

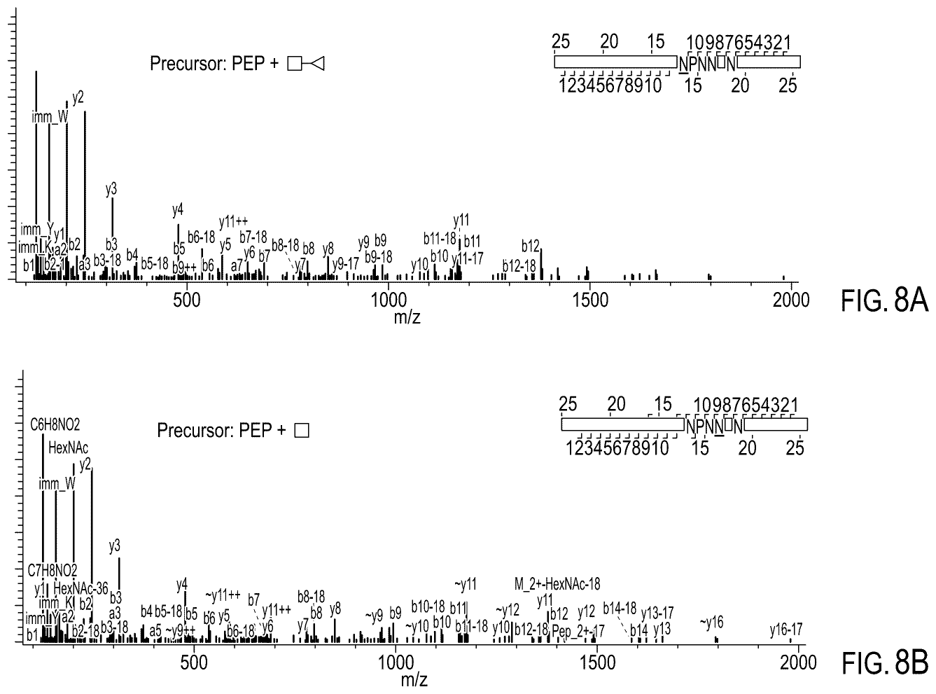

As schematically depicted in FIGS. 8A-8B, the data output of the potential non-consensus N-glycopeptide with BYONIC™, the non-consensus glycosylation site with extensive fragments (mostly b and y ions on the peptide backbone) were successfully identified. Glycopeptides with both glycoforms GlcNAc and GlcNAcFuc were detected after Endo F2 treatment, and the scores of both matches are higher than 900, thereby indicating high-confidence results. The results provided direct evidence supporting the identification and localization of the non-consensus glycosylation at the glycopeptide level. However, the two BYONIC™ matches had inconsistent glycosite assignments on the same tryptic peptide. Simultaneous glycosylation on both sites (Asn 1 and Asn 4, with numbering indicating position from the N-terminus to the C-terminus within the NPNNXN (SEQ ID NO: 1) region), was particularly challenging considering the potential steric hindrance that it may subjected to. Thus, based on the identification in FIGS. 8A-8B, the non-consensus glycosylation site on mAb1 was localized to either Asn 1 or Asn 4.

In-Depth Investigation of Non-Consensus N-Glycosylation in mAb1:

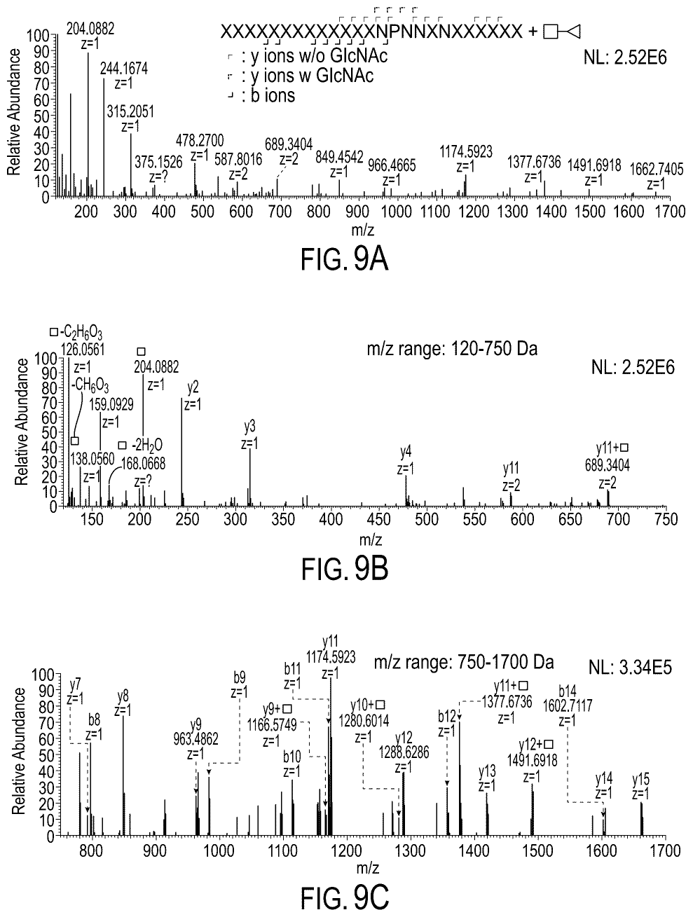

To further pinpoint the non-consensus glycosite on mAb1, the MS2 spectrum of PEP+GlcNAcFuc (FIGS. 9A-9C) was assessed, which was identified as the major glycoform after Endo F2 digestion. FIG. 9A demonstrates a typical MS2 spectrum of a glycopeptide, in which rich fragments were detected at the region of m/z 120-400, whereas more spaced fragments were detected in the high mass range. More specifically, the spectrum was divided into two sections; low mass range (m/z 120-750, FIG. 9B, and high mass range (m/z 750-1700, FIG. 9C. In the low mass range section, multiple GlcNAc-related oxonium ions (peaks at m/z 126, m/z 138, m/z 168, and m/z 204) were identified, thus demonstrating that the selected precursor was glycosylated. Furthermore, various y ions on the peptide backbone of PEP with low m/z values were also detected. In contrast, the high mass range section (FIG. 9C) contained extensive peptide backbone fragments, particularly those covering the glycosites, which are essential for precise localization of the glycosylation site. As schematically depicted in FIG. 9C, the y ions without GlcNAc from y7 to y12 in the NPNNXN (SEQ ID NO: 1) region were detected, except for y10 (detected y ions were annotated according to the key in FIG. 9A. Regarding the y ions with GlcNAc, which can be used to directly assign the glycosite, y9+GlcNAc, y10+GlcNAc, y11+GlcNAc, and y12+GlcNAc were identified, whereas y7+GlcNAc was not detected. The MS2 data suggested that Asn 6 was not glycosylated, whereas Asn 1, Asn 3, and Asn 4 can be glycosylated. Due to the lack of b ions in the NPNNXN (SEQ ID NO: 1) region, it can be challenging to make a conclusive glycosite assignment.

Additional information, such as knowledge of N-glycosylation motif and molecular simulation of the targeted peptide, can aid in resolving such extreme cases. Proline has a unique cyclic structure in the peptide backbone, and its rigid ring structure prevents binding to oligosaccharyltransferases, thus adversely affecting the downstream glycosylation (Valliere-Douglass et al. 2010; 285(21):16012-16022; Taguchi et al. The structure of an archaeal oligosaccharyltransferase provides insight into the strict exclusion of proline from the N-glycosylation sequon. Commun Biol 2021; 4(1):941). As a result, proline at the +1 position of a N-glycosylation motif is a strict exclusion, suggesting that Asn 1 on PEP cannot be glycosylated due to its C-terminal neighboring proline residue.

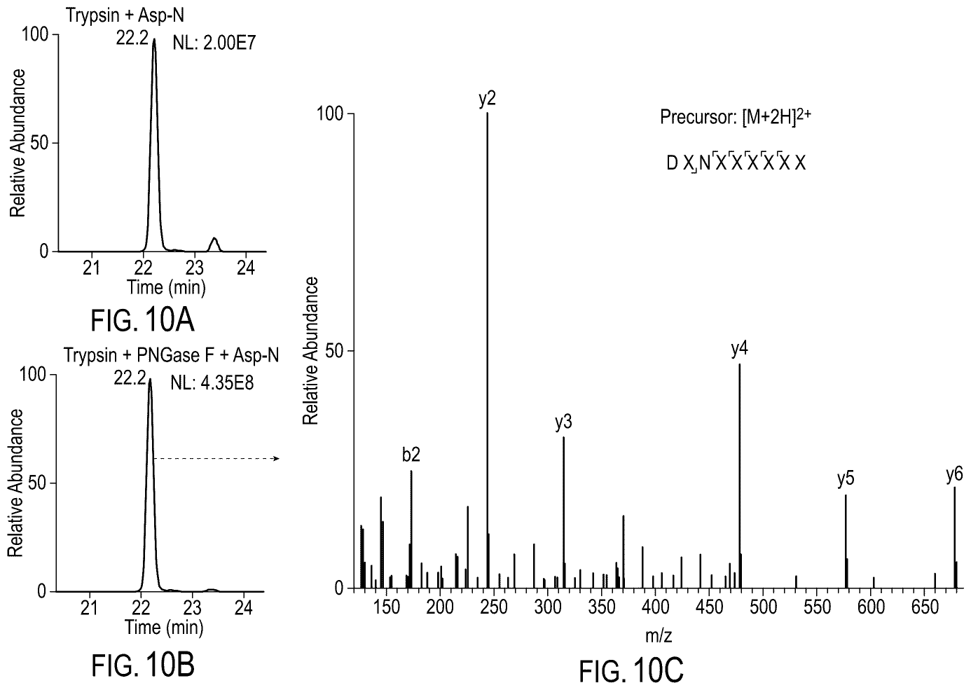

To further validate the glycosite assignment, sequential enzymatic treatments also were performed on mAb1, using trypsin, PNGase F, and Asp-N. After deglycosylation with PNGase F, the original Asn at the glycosylation site was converted into Asp, which provides an additional cleavage site for Asp-N digestion to generate a shorter proteolytic peptide. The EICs and MS/MS spectrum of the product peptide DXNXXXXXX cleaved at Asn 4 is shown in FIGS. 10A to 10C, and multiple corresponding b and y ions were confirmed for the identification (FIG. 10C). The abundance of the product peptide increased by more than 20-fold with PNGase F treatment (FIG. 10B) compared to that of the negative control without PNGase F treatment (FIG. 10A), suggesting that Asn 4 is the non-consensus glycosite on Fab, which is in accordance with the BYONIC™ results using the proposed Endo F2-assisted peptide mapping workflow (FIG. 8B). The presence of DXNXXXXXX in the negative control could be resulted from the endogenous deamidation occurred at Asn 4.

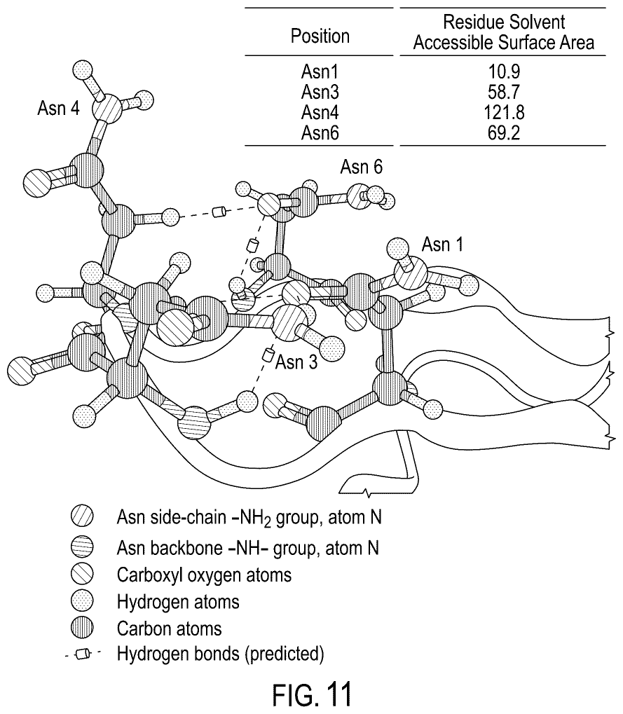

In addition, the in-silico 3D-modeling of mAb 1 with the MOE software package also was performed, specifically on the region covering the NPNNXN (SEQ ID NO: 1) sequence. The calculated SASA indicates the extent of solvent exposure of a residue. Higher SASA values suggest greater possibility of modification. As schematically depicted in FIG. 11, Asn 4 was more flexible and had the largest SASA (more than 1.7-fold compared to that of the other three Asn residue. Potential hydrogen bonding at Asn 3 and Asn 6 can result in a more restricted conformation individually and resulted in less solvent exposure. Asn 1 had the lowest SASA value and therefore was considered to have a low probability for glycosylation, beyond the obstacle of the adjacent proline. The modeling data suggested that Asn 4 (NPNNXN (SEQ ID NO: 1), Asn 4 is bolded and underlined) is the predominant glycosite contributing to the non-consensus N-glycosylation on mAb1, in agreement with the experimental results (FIG. 8B).

After identifying the glycosite, glycosylation occupancy was determined by comparing the EIC peak areas of PEP+GlcNAc/+GlcNAcFuc with the EIC peak area of the non-glycosylated PEP. Since the major glycan portion, especially the negatively charged sialic acid, has been removed with Endo F2, it is reasonable to assume that the discrepancies of ionization efficiencies between peptides and glycopeptides have been minimized after the glycan truncation. Therefore, the ionization efficiencies of PEP, PEP+GlcNAc, and PEP+GlcNAcFuc were considered to be the same, and the calculation was as follows:

$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 100 \%$$

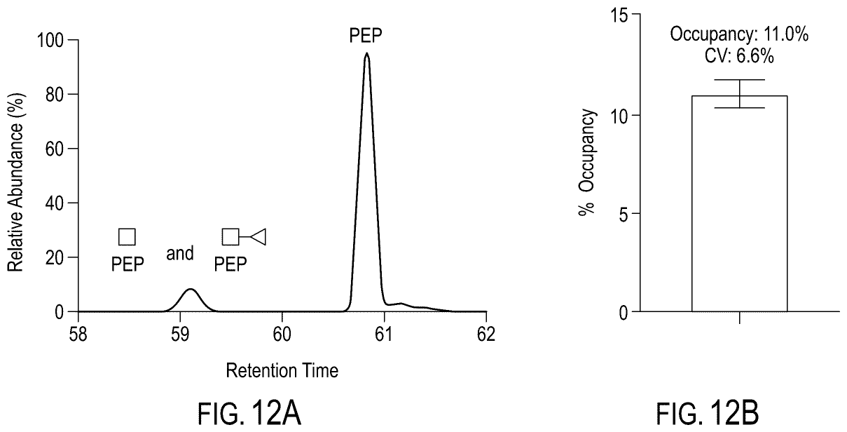

Samples were prepared in triplicate to investigate reproducibility and the reliability of the developed workflow with endoglycosidases. The non-glycosylated PEP and both glycosylated species (PEP+GlcNAc and PEP+GlcNAcFuc) were baseline-separated on a C18 column, and a ˜2 min retention time shift was observed (FIG. 12A). Both glycopeptides completely overlapped in the chromatogram, possibly because the addition of fucose had negligible effects on the hydrophobicity of such a long peptide. On the other hand, the quantitation of the glycosylation occupancy is demonstrated in FIG. 12B. Based on the results from triplicates, the glycan occupancy of PEP was determined to be 11% with excellent reproducibility, as indicated by the assay coefficient of variation (CV) of 6.5%. The major contribution (10%) was from the fucosylated glycoform GlcNAcFuc, whereas the other glycan GlcNAc occupied the remaining 1% thus summing to 11% total glycan occupancy. In addition, the abovementioned quantitation results align well with the nSEC-UV quantitation shown in FIGS. 3A-3C (11.6%), which demonstrates the accuracy and robustness of the developed Endo-F assisted peptide mapping workflow. Furthermore, the calculated relative abundance in the samples without Endo F2 treatment (˜1.3%, FIGS. 4A-4C) was substantially lower than that in the samples with Endo F2 treatment. These discrepancies were expected, because the hydrophilic glycan moiety, particularly the negatively charged sialic acids, could markedly decrease the peptide ionization efficiency. Consequently, the percentage occupancy calculated from the regular tryptic digestion workflow was largely underestimated. Therefore, the partial deglycosylation with Endo F2 facilitated more reliable occupancy quantitation.

The use of Endo F2 for partial deglycosylation provided additional advantages beyond the improved quantitation accuracy. For example, as demonstrated in FIGS. 5A-5B (See SEQ ID NO: 2), the quality of the MS2 spectrum was significantly improved after Endo F2 digestion. In FIG. 5A, without Endo F2 treatment, only oxonium ions generated by the glycan portion were observed, and fragment ions on the peptide backbone were scarcely detected, which hindered the glycosite identification either manually or automatically with software, whereas in FIG. 5B, a more informative spectrum was acquired with Endo F2 treatment. This improvement can be ascribed to the following advantages of Endo F2 digestion: 1) the ionization efficiency of the precursor was increased (both the hydroxyl groups on glycans and carboxyl groups on sialic acids can adversely affect the protonation); 2) the original two glycoforms G2FS and G2FS2 were unified as GlcNAcFuc; and 3) more collision energy was channeled to the peptide backbone due to the truncated glycan moieties. More importantly, the bond between the innermost GlcNAc and the Asn residue was robust enough to remain intact during the fragmentation, thereby enabling the direct assignment of the glycosite (FIG. 9C). Moreover, Asn deamidation is a predominant degradation reaction that can occur in mAbs during production and storage (Lu et al. Deamidation and isomerization liability analysis of 131 clinical-stage antibodies. MAbs 2019; 11(1):45-57). Compared with the conventional complete deglycosylation with PNGase F, which results in artificial deamidation on the glycosylated Asn, the remaining GlcNAc and GlcNAcFuc after partial deglycosylation with Endo F2 generated +203 Da and +349 Da, respectively. Consequently, the resulting glycosylated peptides could be differentiated from the non-glycosylated amidated peptides.

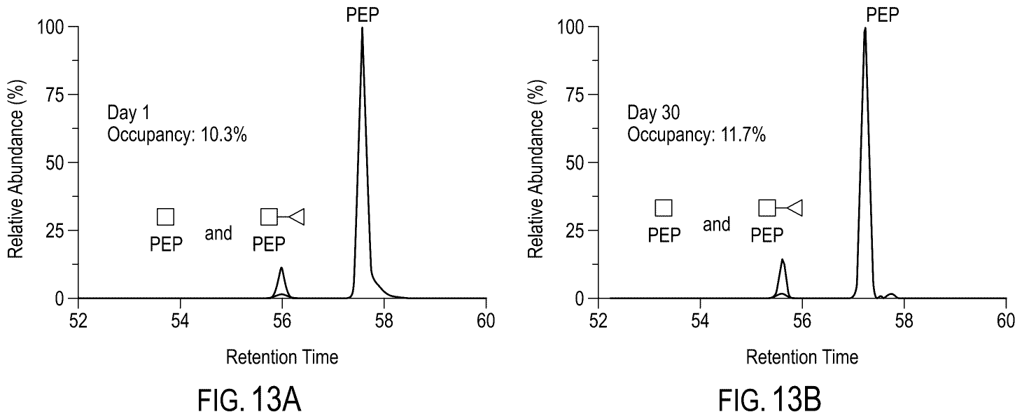

In addition, post-digestion stability regarding the glycosylation occupancy of Endo F2 treated tryptic digests of mAb1 was investigated. Samples were stored in an autosampler set at 4° C. between runs on day 1 and day 30. Results are schematically depicted in FIGS. 13A-13B. Minimal occupancy (1.4%) changes were observed, which could be caused by the instrument variations on different days. The data suggested that the quenched and purified samples after Endo F2 treatment were highly stable, and the occupancy of non-consensus glycosylation was not affected during the one-month storage period.

Application of the Developed Workflow to Other mAbs:

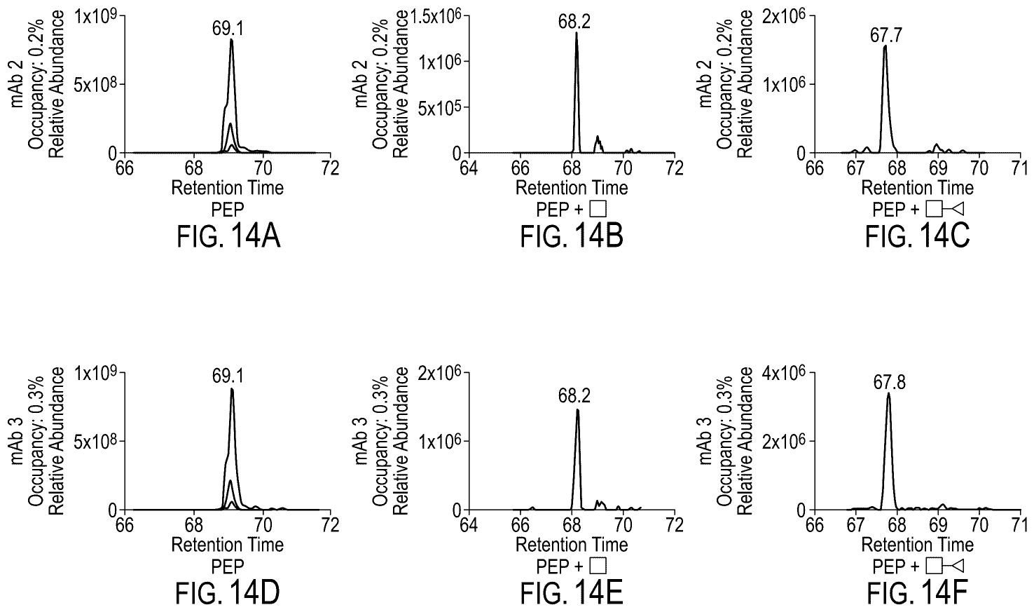

To demonstrate the utility of the developed workflow in quickly localizing potential glycosites and determining the glycosylation occupancy, mAb2 and mAb3 were tested, and were found to have potential for non-Fc-glycosylation, according to the observation of the pre-peak in their nSEC-UV profiles (data not shown). As schematically depicted in FIGS. 14A-14F, atypical N-glycosylation was identified at Asn162 (Kabat numbering, underlined in the following sequence) on a shared peptide, DYFPEPVTVSWNSGALXXXXXXXXXXXXXXXXXXXXXXXXXXXXXXXXX (SEQ ID NO: 4), in the CH1 domain of both mAbs. The identification of the glycosite was in accordance with Valliere-Douglass et al 2009, suggesting that this can be a common non-consensus glycosylation site occurring in mAbs. However, the biological impacts of this modification remain unclear. Furthermore, the glycosylation occupancies were determined to be 0.2% and 0.3% for mAb2 and mAb3, respectively. Such low-abundance glycopeptides (less than 1%) were not detected with the conventional tryptic peptide mapping strategy but were readily identified and quantified with the workflow disclosed herein, thereby indicating the ability of the assay, according to the inventions, to analyze low-abundance non-consensus N-glycosylation.

The inventions demonstrate the feasibility of the developed Endo F-assisted peptide mapping workflow with a challenging case study. The non-consensus Fab glycosylation site located in a 25-residue long tryptic peptide with the sequence of NPNNXN (SEQ ID NO: 1) was identified by the methods disclosed herein. The glycosite was assigned at Asn 4 (the fourth residue within the NPNNXN (SEQ ID NO: 1) sequence) with high confidence, and the glycosylation occupancy was accurately determined. Extensive assessments were performed to validate the software-generated data and the method's reliability. The developed workflow can be applied to other mAbs and successfully identified the non-consensus N-glycosylation with low abundance (about 0.2%). Therefore, the developed workflow can be applied as a useful tool to quickly characterize non-consensus N-glycosylation on mAbs during drug development.

What is claimed is:

1. A system for endoglycosidase-assisted peptide mapping workflow for a protein comprising non-consensus N-glycosylation, wherein the system comprises the steps of:
(a) providing a glycoprotein containing one or more non-consensus N-glycosites;
(b) digesting the glycoprotein using trypsin;
(c) de-glycosylating the digested glycoprotein by treatment with endoglycosidase;
(d) deactivating the endoglycosidase;
(e) desalting the de-glycosylated digested glycoprotein of step (d);
(f) analyzing the purified de-glycosylated digested glycoprotein by nano-flow LC-MS/MS, thereby generating reduced peptide mapping data; and
(g) determining post-translation modifications.

2. The system of claim 1, further comprising validating the peptide mapping data by Extracted Ion Chromatograms (EICs) and MS spectra.

3. The system of claim 1, further comprising identifying glycopeptides in the sample by setting GlcNAc and GlcNAc+Fuc as common variables occurring at Asn and Fc-glycosylation sites.

4. The system of claim 1, further comprising identifying the non-consensus N-glycosylation and quantifying the glycosylation occupancy by data processing that can perform:
(i) screening of glycosylated Asn and glycosite locations,
(ii) analysis of peptide mapping data, and
(iii) determination of post-translation modifications.

5. The system of claim 4, wherein the data processing is performed by a computer, cloud computing, and/or artificial intelligence (AI), and the endoglycosidase is Endo-F2.

6. The system of claim 1, wherein the non-consensus N-glycosite is glycosylated with a glycan selected from the group consisting of G0F, G1F, G2F, G2FS, G2FS1, and G2FS2.

7. The system of claim 1, wherein the non-consensus N-glycosite is located at a NPNNXN (SEQ ID NO: 1) sequence in a 25-residue long tryptic peptide, wherein X can be any amino acid.

8. The system of claim 1, wherein the endoglycosidase-assisted peptide mapping workflow has a detectable glycosylation occupancy of about 0.2%.

9. The system of claim 1, wherein the glycosylation occupancy is calculated as:
$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 1 \; 00 \% .$$

10. The system of claim 1, wherein the protein is selected from the group consisting of an antibody, antibody derivative, antibody fragment, a monoclonal antibody, a monospecific antibody, a bispecific antibody, an Fc-containing protein, and an Fc-fusion protein.

11. The system of claim 1, wherein the de-glycosylated digested glycoprotein from step (c) is desalted by C18 pipette tips.

12. The system of claim 1, wherein the post-translation modification is selected from the group consisting of oxidation, Asparagine (Asn) deamidation, and dehydration.

13. A method of identifying non-consensus N-glycosylation and quantifying glycosylation occupancy in a protein, wherein the methods comprise the steps of
(a) providing a glycoprotein containing one or more non-consensus N-glycosites;
(b) digesting the glycoprotein using trypsin;
(c) de-glycosylating the digested glycoprotein by treatment with endoglycosidase;
(d) deactivating the endoglycosidase;
(e) desalting the de-glycosylated digested glycoprotein of step (c);
(f) analyzing the purified de-glycosylated digested glycoprotein by nano-flow LC-MS/MS, thereby generating reduced peptide mapping data; and
(g) determining post-translation modifications.

14. The method of claim 13 further comprising validating the peptide mapping data by Extracted Ion Chromatograms (EICs) and MS spectra.

15. The method of claim 13 further comprising identifying glycopeptides in the sample by setting GlcNAc and GlcNAc+Fuc as common variables occurring at Asn and Fc-glycosylation site.

16. The method of claim 13 further comprising identifying the non-consensus N-glycosylation and quantifying the glycosylation occupancy by data processing that can perform:
(i) screening of glycosylated Asn and glycosite locations,
(ii) analysis of peptide mapping data, and
(iii) determination of post-translation modifications.

17. The method of claim 16, wherein the data processing is performed by a computer, cloud computing, and/or artificial intelligence (AI), and the endoglycosidase is Endo-F2.

18. The method of claim 13, wherein the non-consensus N-glycosite is glycosylated with a glycan selected from the group consisting of G0F, G1F, G2F, G2FS, G2FS1, and G2FS2.

19. The method of claim 13, wherein the non-consensus N-glycosite is located at a NPNNXN (SEQ ID NO: 1) sequence in a 25-residue long tryptic peptide, wherein X can be any amino acid.

20. The method of claim 13, wherein the endoglycosidase-assisted peptide mapping workflow has a detectable glycosylation occupancy of about 0.2%.

21. The method of claim 13, wherein the glycosylation occupancy is calculated as:
$$\% Occupancy = \frac{Peak {Area}_{( PEP + GlcNAc )} + Peak {Area}_{( PEP + GlcNAcFuc )}}{\begin{matrix}Peak {Area}_{( PEP )} + Peak {Area}_{( PEP + GlcNAc )} + \\ Peak {Area}_{( PEP + GlcNAcFuc )}\end{matrix}} * 1 \; 00 \% .$$

22. The method of claim 13, wherein the protein is selected from the group consisting of an antibody, antibody derivative, antibody fragment, a monoclonal antibody, a monospecific antibody, a bispecific antibody, an Fc-containing protein, and an Fc-fusion protein.

23. The method of claim 13, wherein the de-glycosylated digested glycoprotein from step (c) is desalted by C18 pipette tips.

24. The method of claim 13, wherein the post-translation modification is selected from the group consisting of oxidation, Asparagine (Asn) deamidation, and dehydration.

25. The method of claim 13, wherein the Endo-F2 enzyme is deactivated by acidifying with TFA.

26. A method of identifying non-consensus N-glycosylation and quantifying glycosylation occupancy in a protein, wherein the methods comprise the steps of
(a) de-glycosylating a digested glycoprotein by treatment with endoglycosidase;
(b) deactivating the endoglycosidase;
(c) desalting the de-glycosylated digested glycoprotein of step (b);
(d) analyzing the desalted de-glycosylated digested glycoprotein by nano-flow LC-MS/MS, and thereby generating reduced peptide mapping data; and
(e) determining post-translation modifications.

27. The method of claim 26, further comprising validating the peptide mapping data by Extracted Ion Chromatograms (EICs) and MS spectra.

28. The method of claim 26, further comprising identifying glycopeptides in the sample by setting GlcNAc and GlcNAc+Fuc as common variables occur at Asn and Fc-glycosylation site.

29. The method of claim 26, further comprising identifying the non-consensus N-glycosylation and quantifying the glycosylation occupancy by data processing that can perform:
(i) rapid screening of glycosylated Asn and glycosite locations,
(ii) the analysis of peptide mapping data, and
(iii) determining the post-translation modifications.

30. The method of claim 26, wherein the data processing is performed by a computer, cloud computing and/or artificial intelligence (AI), and the endoglycosidase is Endo-F2.
